SEQUENCE LISTING (13 sequences)

SEQ ID NO: 1 - DNA, 998 bp, Artificial Sequence
  Synthetic construct
atttaggtgacactatagaatgaccctgcttgctcaactctacgtctttgtttcgttttctgttctgcgccgttacagatccaagctgtgaccggcgcctacctgagatcaccggtcaccatggaaatcaaggtgctgtttgccctcatctgtattgctgttgctgaggcaaaacccactgaaatcaatgaagacctcaatatagctgctgtggcctccaactttgccaccacagatcttgagactgacctgttcaccaactgggagaccatgaatgtgattagcactgacacagagcaggtgaacacagatgctgacaggggcaagctgcctggcaaaaaactccccccagatgtcctgagggagctggaggccaatgccagaagggctggttgcacaagaggctgcctcatttgcctctcccacattaagtgcacccctaagatgaagaaatttatccctggcaggtgccacacttatgaaggtgaaaaggagtctgctcagggagggattggagaggcaattgttgatatcccagagattcctggcttcaaggataaggagccactggaccagtttattgctcaagtggacctctgtgctgattgcaccactggctgtctgaagggccttgccaatgtccagtgctctgacctcctgaagaagtggcttccccagaggtgtaccacttttgccagcaagattcagggtagggtggacaaaatcaagggtctggctggggacagatgattagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaaaaaaaaa

SEQ ID NO: 2 - DNA, 1087 bp, Artificial Sequence
  Synthetic construct
atttaggtgacactatagaatgaccctgcttgctcaactctacgtctttgtttcgttttctgttctgcgccgttacagatccaagctgtgaccggcgcctacctgagatcaccggtcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcaggcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaaaaaaaaa

SEQ ID NO: 3 - DNA, 829 bp, Artificial Sequence
  Synthetic construct
atttaggtgacactatagaatgaccctgcttgctcaactctacgtctttgtttcgttttctgttctgcgccgttacagatccaagctgtgaccggcgcctacctgagatcaccggtcaccatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgatagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaaaaaaaaa

SEQ ID NO: 4 - DNA, 1129 bp, Artificial Sequence
  Synthetic construct
atttaggtgacactatagaatgaccctgcttgctcaactctacgtctttgtttcgttttctgttctgcgccgttacagatccaagctgtgaccggcgcctacctgagatcaccggtcaccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaaaaaaaaa

SEQ ID NO: 5 - DNA, 1354 bp, Artificial Sequence
  Synthetic construct
atttaggtgacactatagaatgaccctgcttgctcaactctacgtctttgtttcgttttctgttctgcgccgttacagatccaagctgtgaccggcgcctacctgagatcaccggtcaccatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaaaaaaaaa

SEQ ID NO: 6 - DNA, 10 bp, Artificial Sequence
  Synthetic construct
  misc_feature (4)..(4): R can be A or G
gccrccatgg

SEQ ID NO: 7 - DNA, 12 bp, Artificial Sequence
  Synthetic construct
ccggtcaccatg

SEQ ID NO: 8 - DNA, 23 bp, Bacteriophage T7
taatacgactcactatagggaga

SEQ ID NO: 9 - DNA, 23 bp, Bacteriophage T3
aattaaccctcactaaagggaga

SEQ ID NO: 10 - DNA, 23 bp, Bacteriophage K11
aattagggcacactatagggaga

SEQ ID NO: 11 - DNA, 23 bp, Bacteriophage SP6
atttacgacacactatagaagaa

SEQ ID NO: 12 - DNA, 16 bp, Artificial Sequence
  Synthetic construct
  misc_feature (1)..(6): N can be any nucleoside base
  misc_feature (7)..(7): R can by any purine base (adenosine or guanosine)
  misc_feature (8)..(8): N can be any nucleoside base
  misc_feature (13)..(13): R can by any purine base (adenosine or guanosine)
  misc_feature (15)..(16): N can be any nucleoside base
nnnnnnrncatgrcnn

SEQ ID NO: 13 - DNA, 16 bp, Artificial Sequence
  Synthetic construct
  misc_feature (1)..(3): N can be any nucleoside base
  misc_feature (5)..(5): N can be any nucleoside base
nnngncaccatggcgg

(19) United States
(12) Patent Application Publication
(10) Pub. No.: US 2023/0002799 A1
(43) Pub. Date: Jan. 5, 2023

(54) METHODS OF SYNTHESIZING MRNA AND FUNCTIONAL PROTEINS FROM SYNTHETIC DOUBLE STRANDED DNA

(71) Applicant: Vanderbilt University, Nashville, TN (US)

(72) Inventors: John T. Tossberg, Nashville, TN (US); Thomas M. Aune, Nashville, TN (US)

(21) Appl. No.: 17/784,816

(22) PCT Filed: Dec. 11, 2020
(86) PCT No.: PCT/US2020/064497

§ 371 Date: Jun. 13, 2022

Related U.S. Application Data
(60) Provisional application No. 62/946,561, filed on Dec. 11, 2019.

(51) Int. Cl. C12P 19/34; C12P 21/02; C07K 14/54

(52) U.S. Cl. CPC C12P 19/34; C12P 21/02; C07K 14/5434; C07K 14/5406

(57) ABSTRACT

Disclosed are methods for preparing mRNA and proteins from synthetic DNA.

This application claims the benefit of U.S. Application No. 62/946,561, filed on Dec. 11, 2019, which is incorporated herein by reference in their entirety.

This invention was made with government support under Grant No. AI44924, AI124766, and AI144193 awarded by National Institutes of Health. The government has certain rights in the invention.

### I. BACKGROUND

Since the beginning of modern molecular biology, plasmid based cloning has been an essential component of both basic and medical research. A limitation is that plasmid-based cloning is a complicated, multi-step process. DNA needs to be isolated and plasmid-based vectors prepared. DNA fragments are ligated into plasmid-based vectors and transformed into bacteria. Finally, bacteria are plated and screened to identify the correct clone expressing the DNA construct of interest. The entire process takes many days if not weeks and even then, there is no guarantee that the correct clone will be identified. Not only is the process time consuming, it is also expensive. Vectors, restriction enzymes, ligases, Taq polymerase, competent cells and bacterial plates are just some of the components required and these can cost thousands of dollars.

With advances in vitro transcription (IVT) technology from either plasmid based DNA templates or DNAs PCR-amplified from plasmids, as well as in delivery vehicles including polymeric- and lipid-based nanoparticles, interest has increased in the use of RNA molecules, including mRNA molecules as therapeutics for human disease. Examples include loss-of-function mutations such as adenosine deaminase deficiency, ornithine transcarbamylase deficiency, phenylketonuria, haemophilia B, and cystic fibrosis. Use of mRNAs is also gaining in interest as an alternative to attenuated viruses, inactivated viruses or protein sub-units in development of vaccines. Examples of different mRNA candidates for either replacement therapies for diseases arising from inactivating mutations or as vaccine candidates have all advanced to various stages of preclinical or clinical development as therapeutics for their target diseases. In fact, one of five of the confirmed Covid-19 vaccine candidates that had reached the stage of clinical development by the end of April, 2020 employed a mRNA-based platform for vaccination against this novel coronavirus. mRNAs are also attractive candidates for a variety of therapeutic applications due to their low toxicity, low immunogenicity, cost of production as well as other factors compared to more traditional platforms. For the above reasons, use of mRNAs as therapeutic modalities may increase substantially in the future. What are needed are improvements in costs and ease of mRNA and protein synthesis.

### II. SUMMARY

Disclosed are methods related to generating RNA or proteins from synthetic DNA.

In one aspect, disclosed herein are methods of making a synthetic ribonucleic acid (RNA) strand, the method comprising obtaining a double stranded (ds) deoxyribonucleic acid (DNA) (such as, for example a synthetic dsDNA) comprising a nucleic acid of interest (such as, for example a gene of interest) and transcribing RNA from the dsDNA in vitro. In some aspects, the dsDNA comprises in order from 5′ to 3′ an RNA promoter sequence (such as, for example, a DNA dependent RNA polymerase promoter sequence including, but not limited to the T7 promoter (SEQ ID NO: 8), T3 promoter (SEQ ID NO: 9), K1 promoter (SEQ ID NO: 10), or SP6 promoter (SEQ ID NO: 11)), a 5′ untranslated region (UTR), a Kozack sequence (such as, for example, CCGGTCACCATG (SEQ ID NO: 7) or GCCRCCATGG (SEQ ID NO: 6), the nucleic acid of interest, and a 3′ UTR.

Also disclosed herein are methods of making a synthetic RNA strand of any preceding aspect, further comprising adding a 5′ CAP to the transcribed RNA and/or adding a polyAdenosine (polyA) tail to the 3′ end of the transcribed RNA.

In one aspect, disclosed herein are methods of making a synthetic RNA strand of any preceding aspect, wherein the nucleic acid of interest is between 100 and 10,000 base pairs in length.

Also disclosed herein are methods of making an exogenous protein from a synthetic deoxyribonucleic acid (DNA) comprising obtaining a double stranded (ds) deoxyribonucleic acid (DNA) (such as, for example a synthetic dsDNA) comprising a nucleic acid of interest, transcribing ribonucleic acid (RNA) from the dsDNA in vitro, and transfecting a cell with the transcribed RNA; wherein the transfected RNA is expressed by the cell. In some aspects, the dsDNA comprises in order from 5′ to 3′ an RNA promoter sequence (such as, for example, a DNA dependent RNA polymerase promoter sequence including, but not limited to the T7 promoter (SEQ ID NO: 8), T3 promoter (SEQ ID NO: 9), K1 promoter (SEQ ID NO: 10), or SP6 promoter (SEQ ID NO: 11)), a 5′ untranslated region (UTR), a Kozack sequence (such as, for example, CCGGTCACCATG (SEQ ID NO: 7) or GCCRCCATGG (SEQ ID NO: 6)), the nucleic acid of interest, and a 3′ UTR.

In one aspect, disclosed herein are methods of making an exogenous protein from a synthetic deoxyribonucleic acid (DNA) of any preceding aspect, further comprising adding a 5′ CAP to the transcribed RNA and/or adding a polyAdenosine (polyA) tail to the 3′ end of the transcribed RNA prior to transfection.

Also disclosed herein are methods of making an exogenous protein from a synthetic deoxyribonucleic acid (DNA) of any preceding aspect, wherein the nucleic acid of interest is between 100 and 10,000 base pairs in length.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows analysis of addition of poly (A) tail to synthetic RNAs by agarose gel electrophoresis. From left to right, the 1-kb ladder and the indicated synthetic RNAs before (−) and after (+) enzymatic addition of the poly (A) tail. eGFP: Enhanced green fluorescent protein.

FIG. 2 shows a schematic describing production of synthetic genes and mature mRNAs. Synthetic genes were designed with, from 5′ to 3′, the SP6 bacteriophage promoter, a 5′UTR, Kozak sequence, coding sequence of interest and 3′ UTR and transcribed into RNA. A 5′ Cap and poly (A) tail were enzymatically added to the purified single-stranded RNA to yield the mature mRNA.

FIGS. 3A, 3B, 3C, and 3D show functional proteins generated from synthetic genes. FIG. 3A shows protein yields produced by HeLa cells transfected with the indicated mRNAs produced from synthetic genes; secreted luciferase (determined by measuring enzymatic activity), IL4 and IL12 (determined by ELISA). FIG. 3B shows the expression of eGFP by HeLa cells transfected with eGFP mRNA determined by fluorescence microscopy. FIG. 3C shows the indicated fractions of supernatant fluids from HeLa cell cultures transfected with IL4 mRNA or purified recombinant IL-4 were added to PBMC cultures stimulated with anti-CD3 and anti-CD28. After five days of culture, supernatant fluids were harvested and IL-5 protein levels determined by ELISA. * P<0.05 compared to mock transfected cultures. FIG. 3D shows the indicated fractions of supernatant fluids from HeLa cell cultures transfected with IL12A and IL12B mRNAs or purified recombinant IL-12 were added to PBMC cultures stimulated with anti-CD3 and anti-CD28. After five days of culture, supernatant fluids were harvested and IFN-g protein levels determined by ELISA. * P<0.05 compared to mock transfected cultures.

FIGS. 4A and 4B show synthetic mRNA amount and time dependence upon luciferase protein expression. FIG. 4A shows the indicated amounts of luciferase mRNA (o) or un-capped and un-poly (A) tailed luciferase RNA ( ) were transfected into HeLa cells. Culture supernatant fluids were harvested after 24 hr. and assayed for luciferase activity. FIG. 4B shows HeLa cells were transfected with 100 ng luciferase synthetic mRNA. Culture supernatant fluids were harvested at the indicated times and assayed for luciferase activity.

FIG. 5 shows a graphical representation of the consensus sequence motif for the Kozack sequence.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF MAKING RNA OR PROTEINS

Since the beginning of modern molecular biology, plasmid based cloning has been an essential component of research. Cloning is a complicated, multi-step process. DNA needs to be isolated and plasmid-based vectors need to be prepared. These DNA fragments are then ligated and transformed into bacteria. Finally, bacteria are plated out and screened to find the correct clone. The entire process takes many days if not weeks and even then there is no guarantee that the correct clone will be identified. Not only is the process time consuming, it is expensive. Vectors, restriction enzymes, ligases, Taq polymerase, competent cells and bacterial plates are just some of the components required and these can cost thousands of dollars.

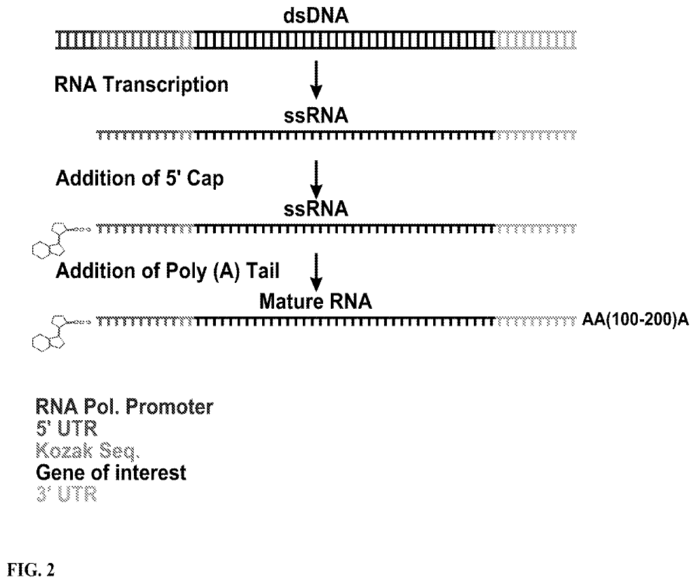

Recently, advances in the chemical synthesis of DNA have significantly lowered the cost and increased the length of the DNA that can be purchased commercially. It is now possible to purchase or synthesize a double stranded DNA (dsDNA) molecule that is 3000 base pairs in length for about $550.00. Due to these advances, the unique process disclosed herein offers a faster and significantly cheaper way to express RNAs and proteins. Basically, a double stranded DNA containing the gene or sequence of interest can be purchased or synthesized. Additional sequences can be included as well. These are in order from 5′ to 3′, the RNA promoter sequence, 5′ UTR, Kozack sequence, the nucleic acid of interest (e.g., gene of interest), and finally a 3′ UTR. Once the DNA arrives it is re-suspended in water and a portion of it is used as a template for the RNA transcription reaction. This is allowed to incubate overnight. DNAse is added to remove the template DNA. The RNA is precipitated and quantified. Next, RNA is used for the subsequent capping and poly (A) tailing reactions. At this point the mature RNA is ready to be transfected into cells (See FIG. 2). The entire process can be completed in two days, and in terms of materials and time is significantly less expensive than plasmid-based cloning.

The process disclosed herein offers a unique method to express RNAs and proteins. It is significantly faster and less expensive than currently available methods. It also affords a level of experimental control, in terms of knowing the exact amount of RNA being transfected into a cell. The process disclosed herein leapfrogs the plasmid cloning aspect of the process by going directly to the mature RNA. If one was using plasmid-based cloning there is no reason to construct a mature RNA, it is an extra step and in the vast majority of cases, unnecessary. We believe that this process represents a potentially disruptive technology that can radically change the plasmid based cloning industry.

Accordingly, in one aspect, disclosed herein are methods of making a synthetic ribonucleic acid (RNA) strand (such as, for example messenger RNA (mRNA)), the method comprising obtaining a double stranded (ds) deoxyribonucleic acid (DNA) (such as, for example a synthetic dsDNA) comprising a nucleic acid of interest (such as, for example a gene of interest) and transcribing RNA from the dsDNA in vitro (for example, using a using a polymerase appropriate for the DNA dependent RNA polymerase promoter used in the presence of nucleotides (i.e., ATP, GTP, CTP, and UTP)). It is understood and herein contemplated that once the RNA is transcribed, it may be desirous to express the encoded protein. Thus, also disclosed herein are methods of making an exogenous protein from a synthetic deoxyribonucleic acid (DNA) (such as, for example a synthetic dsDNA) comprising obtaining a double stranded (ds) deoxyribonucleic acid (DNA) comprising a nucleic acid of interest, transcribing ribonucleic acid (RNA) from the dsDNA in vitro, and transfecting a cell with the transcribed RNA; wherein the transfected RNA is expressed by the cell.

It is understood and herein contemplated that the disclosed nucleic acids for use in the disclosed methods of synthesizing RNA or proteins can also comprise a DNA dependent RNA polymerase to drive expression of the RNA during transcription. Preferably, the RNA promoter is a DNA dependent RNA polymerase promoter. DNA dependent RNA polymerase promoters can be obtained from any source of such promoters including, but not limited to bacteriophage such as, for example, Examples of bacteriophage comprises DNA dependent RNA polymerases include T7, T3, K11, SP6, ϕ29, P22, X phage, T4, Mu, P1, P2, T5, HK97, N15, and FLIP. Thus, in one aspect, the RNA promoter comprises a DNA dependent RNA polymerase promoter sequence including, but not limited to the T7 promoter (SEQ ID NO: 8), T3 promoter (SEQ ID NO: 9), K1 promoter (SEQ ID NO: 10), or SP6 promoter (SEQ ID NO: 11)), It is understood that the promoter used should be appropriate for the DNA dependent RNA polymerase used to during the transcription reaction. Thus, for example, where the promoter used is the T7, the polymerase should be the T7 polymerase; where the promoter used is the T3, the polymerase should be the T3 polymerase; where the promoter used is the K11, the polymerase should be the K11 polymerase; and where the promoter used is the SP6, the polymerase should be the SP6 polymerase.

The dsDNA used in the disclosed methods can contain a Kozack sequence. As used herein, the Kozack sequence comprises at least 10 contiguous nucleic acids and the start site (ATG) of the motif NNNNNNRNCATGRCNN (SEQ ID NO: 12), where R can by any purine base (adenosine or guanosine), Y can be any pyrimidine base (i.e., cytosine or thymine), and N can be any nucleoside base. Thus, in one aspect the Kozack sequence comprises 10 contiguous nucleic acids of the motif NNNGNCACCATGGCGG (SEQ ID NO: 13). For example, the Kozack sequence can comprise the sequence, CCGGTCACCATG (SEQ ID NO: 7) or GCCRCCATGG (SEQ ID NO: 6). The Kozack sequence can be ordered as part of a larger dsDNA, synthesized with the dsDNA or added to dsDNA prior to transcription. Thus, disclosed herein are methods making a synthetic ribonucleic acid (RNA) strand or protein, wherein the dsDNA comprises a Kozack sequence (such as, for example, CCGGTCACCATG (SEQ ID NO: 7) or gccRccATGG (SEQ ID NO: 6)).

As noted throughout the specification, to be successfully synthesized and ultimately expressed, the dsDNA comprises in order from 5′ to 3′ an RNA promoter sequence (such as, for example, a DNA dependent RNA polymerase promoter sequence including, but not limited to the T7 promoter (SEQ ID NO: 8), T3 promoter (SEQ ID NO: 9), K1 promoter (SEQ ID NO: 10), or SP6 promoter (SEQ ID NO: 11)), a 5′ untranslated region (UTR), a Kozack sequence (such as, for example, CCGGTCACCATG (SEQ ID NO: 7) or gccRccATGG (SEQ ID NO: 6)), the nucleic acid of interest, and a 3′ UTR.

It is understood and herein contemplated that for successful translation of the synthesized RNA (such as mRNA) to occur a 5′ CAP and/or 3′ polyAdenosine (polyA) tail can be added to the transcribed RNA. The 5′ cap can be any suitable nucleotide but is preferably guanine or a guanine variant linked to the RNA in a 5′ to 5′ linkage. The polyadenylation of the transcribed RNA can comprise the addition of any polyA tail comprising up to 25 adenosine monophosphates.

The disclosed methods for synthesizing a RNA (such as, for example mRNA) or a protein can work with any length of any nucleic acid that can be synthesized or purchased. Thus, the only real limit on the size of the nucleic acid is the ability to synthesize or purchase said nucleic acid. Thus, disclosed herein are methods of making a synthetic RNA strand, wherein the nucleic acid of interest (i.e., the gene of interest) is between 100 and 10,000 base pairs in length. For example, the nucleic acid of interest (i.e., the gene of interest) can be between 500 and 3000 base pairs in length, or between 500 and 1500 base pairs in length. For example, the nucleic acid of interest (i.e., the gene of interest) can be at least 100, 200,300, 400, 500, 600, 700, 800, 900, 1000, 1100, 1200, 1300, 1400, 1500, 2000, 2500, 3000, 3500, 4000, 4500, 5000, 5500, 6000, 6500, 7000, 7500, 8000, 8500, 9000, 9500, or 10,000 base pairs.

RNA transcription can occur through any means known in the art including use of kits (for example, the MEGAscript SP6 kit, MEGAscript T7 kit, HiScribe T7 kit). Briefly, the DNA constructed is heated and cooled. The nucleotides, reaction buffer, and the appropriate RNA polymerase matching the DNA dependent RNA polymerase promoter is added. The reaction is incubated after which DNAse is added and the RNA precipitated.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

4. Nucleic Acids

There are a variety of molecules disclosed herein that are nucleic acid based. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the RNA and/or protein synthesis disclosed herein, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

5. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the nucleic acid of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson. Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et at, Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from transfected RNA in mammalian host cells may be obtained from various sources, for example, DNA dependent RNA polymerase promoters from the genomes of bacteriophage such as the T7 (SEQ ID NO: 8), T3 (SEQ ID NO: 9), K11 (SEQ ID NO: 10), or SP6 (SEQ ID NO: 11) bacteriophage promoters. It is understood and herein contemplated that any other DNA dependent RNA polymerase promoter from any bacteriophage can be obtained and used in the disclosed methods. Examples of bacteriophage comprises DNA dependent RNA polymerases include ϕ29, P22, λ phage, T4, Mu, P1, P2, T5, HK97, N15, and FLIP.

or viruses such as polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DM-1k− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

7. Peptides

a) Protein Variants

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage For example, linkages for amino acids or amino acid analogs can include CH₂NH—, —CH₂S—, —CH₂—CH₂—CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: A Simplified Method to Produce mRNAs and Functional Proteins from Synthetic Double-Stranded DNA Templates

a) Methods

### (1) Cell Culture

Hela cells (ATCC, CCL-2) were cultured in RPMI 1640 (Gibco, cat. no. 21870) supplemented with 10% (vol/vol) fetal calf serum (Atlanta Biologicals cat. no. S12450), 100 U/ml penicillin 100 μg/ml streptomycin and 2 mM L-Glutamine (final concentration, Gibco, cat. no. 15140). Cells were cultured at 37° C. in the presence of 5% CO2 in humidified air.

Human PBMC were isolated from healthy volunteers and stimulated with soluble mouse anti-human CD28 (1 μg ml⁻¹; 555725; BD Biosciences) and plate-bound anti-CD3 (10 μg ml⁻¹; OKT3 clone, American Type Culture Collection), 1×10⁶ cells ml⁻¹, under TH1 polarizing conditions: IL-12 (10 ng ml⁻¹), TH2 polarizing conditions (IL-4 (10 ng ml⁻¹) or non-polarizing conditions; no additional cytokines. Cultures fluids were harvested after 5 days and analyzed for expression of IFN-γ or IL-13 by ELISA. The study was approved by the institutional review board at Vanderbilt University. Written informed consent was obtained at the time of blood sample collection.

### (2) Constructs

Double stranded DNA molecules (gBlocks) were ordered from Integrated DNA Technologies (IDT). DNA constructs were designed with the SP6 RNA promoter sequence, 5′ UTR, Kozak sequence and ATG initiation sequence at the 5′ end followed by the gene insert. A 3′ UTR and short poly (A) sequence were added at the 3′ end of the double stranded DNA molecule. Samples were resuspended in RNase, DNase free distilled water (Invitrogen, cat. no. 10977) to a concentration of 20 ng/μl. Samples were heated at 50° C. for 20 minutes as per manufactures instruction and stored at −20° C.

### (3) RNA Transcription

RNA transcription was performed using the Megascript SP6 kit (Invitrogen, cat. no. AM1330) according to manufacturer's instructions. Briefly, 8 μl of DNA construct were heated for 5 minutes at 65° C. and cooled. 2 μl of ATP, CTP, GTP UTP, 10× reaction buffer and SP6 RNA polymerase were added. The reaction was incubated overnight at 37° C. The next day 1 μl of Turbo DNAse was added and incubated for 30 minutes. The reaction was stopped by addition of 30 μl of dH20 and 30 μl of lithium chloride precipitation solution. The reaction was incubated for 30 minutes at −20° C., followed by centrifugation at 4° C. at 14000 RPM, 20000 g for 15 minutes. The supernatant was removed and pellet washed with 1 ml 70% ethanol. The centrifugation step was repeated once under the same conditions. The 70% ethanol was removed and the pellet briefly air-dried prior to suspension in 50 μl nuclease free water. Absorbance was determined at 260 nm to quantitate yields of RNA and stored at −80° C. Yields typically totaled ˜10-70 μg.

### (4) 5′ Capping Reaction

The capping reaction was performed using the Vaccinia Capping System (New England Biolabs, cat. no. 2080S) following the manufacturer's instructions and capping efficiency is estimated to exceed 95%. Briefly, 10 μg of RNA was combined with nuclease free water to a final volume of 15 μl and heated to 65° C. for 5 minutes. The tube was cooled on ice for 5 minutes. The following kit ingredients were added: 2 μl 10× capping buffer, 1 μl 10 mM guanosine triphosphate (GTP), 1 μl 2 mM S-adenosylmethionine, 2 μl Vaccinia Capping Enzyme.

This reaction was incubated for 30 minutes at 37° C.

### (5) RNA Cleanup

After the capping reaction, the RNA was cleaned up using the Qiagen RNeasy MinElute Cleanup kit (Qiagen cat. no. 74204) following the manufacturer's instructions.

### (6) Poly (A) Tailing Reaction

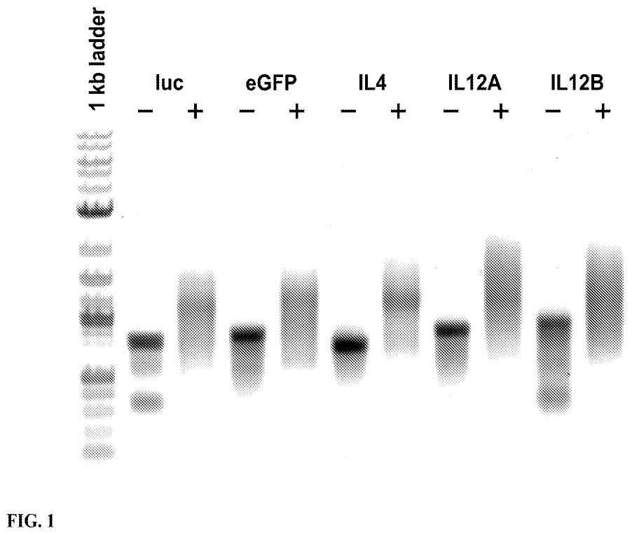

The addition of a poly (A) tail was performed using E. coli Poly (A) polymerase (New England Biolabs, cat. no. M276S), following the manufacturer's instructions. Briefly, up to 10 μg of capped RNA was diluted to 15 μl using nuclease free water. To the RNA, 2 μl of 10× E. coli Poly (A) polymerase reaction buffer, 2 μl 10 mM adenosine triphosphate (ATP) and 1 μl E. coli Poly (A) polymerase (5 units) were added and incubated for 30-60 minutes at 37° C. The capped and tailed RNA was purified using the RNA cleanup protocol described above and quantitated. Analysis of the synthetic mRNA by agarose gel electrophoresis demonstrated efficiency of poly (A) tail addition was >95% (FIG. 1).

### (7) Transfections

Transfections were performed using Lipofectamine® RNAiMAX transfection reagent (ThermoFisher Scientific, cat. no. 13778150) according to manufacturer's instructions. All dilutions were performed in Opti-MEM medium (ThermoFisher Scientific, cat. no. 31985070). Briefly, on day 0, Hela cells were plated at 100,000 cell/ml and in either 96 or 6 well plates at volumes of either 100 μl or 3 ml, respectively. On day 1, 1.5 μl of RNAiMAX was diluted into 25 μl of Opti-MEM. This was scaled depending upon the number of samples, dilutions and culture volumes. Mature mRNAs were diluted in Opti-MEM at varying concentrations before addition to cell cultures. Culture after transfection was typically 24 hr. Transfection experiments were performed a minimum of three times. Unpaired t-tests with Welch's correction were employed to determine statistical significance.

b) Results

We designed synthetic double-stranded DNAs with the bacteriophage SP6 promoter at the 5′ end followed by a synthetic 5′ untranslated region (UTR), a Kozak sequence, transcriptional start site, complimentary DNA to the mRNA of interest, and a 3′ UTR (obtained from IDT). We performed in vitro transcription reactions to synthesize single-stranded RNA. Yields of RNA from 100 ng of DNA were typically 100-300 μg. The 5′ cap and poly (A) tail were added to the RNA (FIG. 2). Purified mRNAs were transfected into target cells and protein expression and function determined as outlined below.

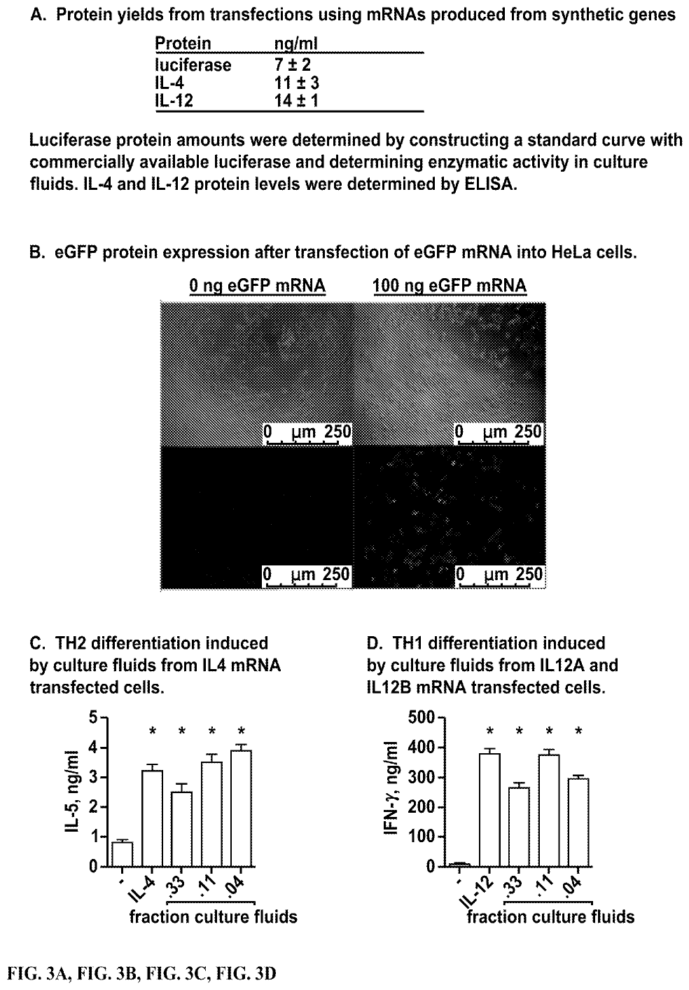

Using the above process, we designed a synthetic gene to express a secreted form of luciferase. The coding sequence we inserted into the synthetic gene was 621 bp. The rationale for choosing the luciferase gene was that luciferase is an oxidoreductase enzyme. The enzymatic reaction requires molecular oxygen and reduced flavin to catalyze light emission. We assumed that accurate transcription, translation, folding and entry into secretory pathways would all be required to produce secreted luciferase that could be detected in cell culture supernatant fluids. Varying amounts of the synthetic luciferase mRNA were transfected into HeLa cells. After 24 hrs, culture fluids were harvested and luciferase activity measured. Abundant expression of luciferase protein activity was found in cultures transfected with 100 ng/well of the synthetic luciferase mRNA (FIG. 3A).

Using the same process the next synthetic gene we prepared was enhanced green fluorescent protein (eGFP). The coding sequence we inserted into the synthetic gene was 718 bp. The rationale was that for emission of green fluorescence from cells, eGFP must be accurately transcribed, translated, form a homodimer, and remain in the intracellular space. We transfected 100 ng of eGFP mRNA into HeLa cells and determined eGFP protein expression by fluorescence microscopy. We found abundant expression of green fluorescence in eGFP mRNA transfected HeLa cells but not in mock transfected HeLa cells (FIG. 3B). After transfection of cultures with the eGFP mRNA, we found that ˜50% of cells were eGFP+ indicating that transfection efficiency was at least 50%. We conclude that the synthetic eGFP gene was accurately transcribed in the in vitro transcription reaction, translated and folded properly inside cells to produce active eGFP protein.

IL-4 and IL-12 are cytokines that play critical roles in both the innate and adaptive arms of the immune response. Perhaps most notably, IL-4 directs differentiation of naïve T cells into effector T helper 2 (TH2) cells capable of producing the cytokines, IL-4, IL-5, and IL-13, critical to control extracellular parasite infections by the adaptive arm of the immune system and IL-12 directs the differentiation of naïve T cells into effector T helper 1 (TH1) cells to enable their expression of IFN-γ, a critical cytokine required for protection against an array of intracellular pathogens. We designed an IL4 synthetic gene. The IL4 coding sequence was 456 nt. The IL4 synthetic mRNA, prepared by the above method, was transfected into HeLa cells (100 ng/culture) and culture fluids harvested after 24 hr. IL-4 protein levels in mock transfected and synthetic IL4 mRNA transfected cultures were determined by ELISA (FIG. 3A). Biological activity of IL-4 produced from the IL4 mRNA was determined in a TH2 differentiation assay. Briefly, human peripheral blood mononuclear cells (PBMC) were treated with anti-CD3 and anti-CD28 to stimulate T cell proliferation and either purified IL-4 or culture fluids from HeLa cells transfected with IL4 mRNA. Culture fluids from PBMC cultures were harvested after 5 days and IL-5 levels determined by ELISA as a measure of TH2 differentiation. We found that levels of IL-4 produced from IL4 mRNA induced substantial TH2 differentiation as determined by the ability of naïve human T cells to differentiate into effector TH2 cells that express IL-5 protein (FIG. 3A, C). Active IL-12 is composed of a heterodimeric protein consisting of IL-12 p35 and IL-12 p40 subunits, also named IL-12A and IL-12B, respectively. We designed both IL12A and IL12B synthetic genes using the above processes. The IL12A coding insert was 759 bp and the IL12B coding insert was 984 bp. Both IL12A and IL12B mRNAs were simultaneously transfected into HeLa cells, 100 ng/well. Culture fluids were harvested after 24 hr. IL-12 protein levels were determined by enzyme-linked immunosorbent assay (ELISA) (FIG. 3A). Ability to induce TH1 differentiation was determined by stimulating human peripheral blood mononuclear cells (PBMC) with anti-CD3 and anti-CD28 and either purified IL-12 or culture fluids from HeLa cells transfected with IL12A and IL12B mRNAs. Culture fluids from PBMC cultures were harvested after 5 days and IFN-γ levels determined by ELISA as a measure of TH1 differentiation. We found that culture fluids from IL12A and IL12B mRNA transfected HeLa contained abundant levels of IL-12 protein while IL-12 protein was undetectable in mock transfected HeLa culture fluids (FIG. 3A). We also found that culture fluids from IL12A and IL12B mRNA transfected HeLa cells were potent inducers of PBMC TH1 differentiation as determined by the ability of these culture fluids to induce expression of IFN-γ by PBMC cultures (FIG. 3D). Thus, these results indicate that IL4 synthetic genes and IL12A and IL12B synthetic genes were efficiently transcribed into mRNA and formed biologically active proteins capable of inducing TH2 or TH1 differentiation, respectively.

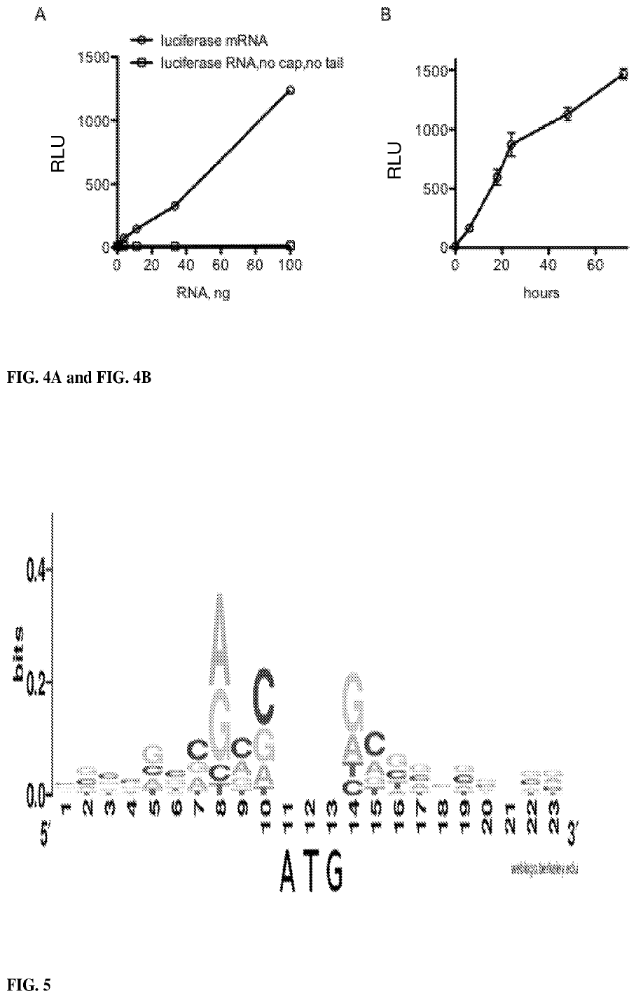

We also varied the amount of luciferase synthetic mRNA transfected into cultures and assayed presence of luciferase in the cultured fluid. We found that yield of luciferase was proportional to the amount of transfected luciferase mRNA over a range of 1-100 ng/culture (FIG. 4A). In contrast, transfection of luciferase synthetic RNA without addition of the 5′ CAP and poly (A) tail did not result in production of detectable luciferase protein indicating these steps were necessary to produce a functional mRNA. We also compared yield of luciferase protein as a function of time after transfection with synthetic luciferase mRNA. We found that levels of secreted luciferase steadily increased over at least a 72-hour period (FIG. 4B). Thus production of luciferase protein was proportional to both amount of transfected synthetic mRNA and time of culture.

c) Discussion

In conclusion, we present a simple, inexpensive and rapid method to prepare synthetic genes and mature mRNAs that can be efficiently introduced into cells and translated into functional proteins without the need for plasmid-based cloning (Table 1). Other RNAs, such as long noncoding RNAs, can also be prepared using this method. These synthetic genes can have many cell-based applications, such as structure-function studies or studies akin to site-directed mutagenesis studies, gain or recovery of function studies. These mRNAs produced from synthetic genes can also have applications in vivo. For example, with newer delivery vehicles, such as nanoparticles, these mRNAs can have medical applications, such as gene therapy or vaccine development without the need for plasmid-based vectors or viral delivery vehicles.

TABLE 3
Cost and time estimates for synthetic RNA versus plasmid-based cloning methods

| | | | Reagent | Cost for | Hands on | Total |
|---|---|---|---|---|---|---|
| | | Vendor | costs ($) | 1 ($) | time (hrs) | time (hrs) |
| | Synthetic mRNA | | | | | |
| Day 1 | Design gBLOCK with SP6 promoter | IDT | 75-3000 | 150.0 | 1 | 2 weeks |
| Day 2 | In vitro Transcription/LiCl ppt | Megascript | 8.6/rxn | 8.60 | 2 | 14 |
| Day 3 | Add 5′ Cap | NEB | 3.5/rxn | 3.50 | 0.5 | 2 |
| Day 3 | Add poly A tail | NEB | 3.6/rxn | 3.60 | 0.5 | 1 |
| Day 3 | RNA clean up 2X | Qiagen | 7.38/rxn | 14.76 | 1 | 1 |
| | Totals | | | 180.46 | 5 | 18 |
| | Traditional plasmid-based cloning | | | | | |
| Day 1 | Design gBLOCK with restriction sites. | IDT | 75.00-3000 | 150.00 | 1 | 2 weeks |
| Day 1 | Purchase Expression Vector | NEB | 100/clone | 100.00 | 1 | 48 |
| Day 2 | Digest vector and gBLOCK | NEB | .05/u | 0.10 | 0.5 | 1 |
| Day 2 | Run on gel | Sigma | 1.97/gr | 1.97 | 0.5 | 1 |
| Day 2 | Gel extraction + clean-up | Qiagen | 2.3/rxn | 2.30 | 3 | 3 |
| Day3 | Ligation | Thermofisher | 1.18/rxn | 1.18 | 0.5 | 0.5 |
| Day3 | Transfomation | Thermofisher | 19.92/rxn | 19.92 | 1 | 1 |
| Day3 | Out grow | media | negligible | | 0.5 | 18 |
| Day3 | Plating | media | 2/plate | 10.00 | 1 | 2 |
| Day 4 | Colony screening | Taq | .5/U | 10.00 | 1 | 18 |
| Day 4 | Run on gel | Sigma | 1.97/gr | 3.94 | 0.5 | 2 |
| Day 4 | Pick PCR positives, start cultures | media | negligible | | 1 | 18 |
| Day 5 | Make mini preps | Qiagen | 1.81/rxn | 23.60 | 4 | 3 |
| Day 5 | Digest with RE | Neb | .05/U | 1.00 | 0.5 | 1 |
| Day 5 | Run on gel | Sigma | 1.97/gr | 3.94 | 1 | 2 |
| Day 6 | Send clone for sequencing validation | Genewiz | 4/rxn | 20.00 | 1 | 18 |
| Day 7 | Start cultures for Maxi preps. | media | negligible | | 0.5 | 18 |
| Day 8 | Prepare DNA for transfections | Qiagen maxi | 24.5/rxn | 24.50 | 4 | 18 |
| | Totals | | | 372.45 | 22.5 | 172.5 |

*gBLOCK fragment price versus nucleotide length

| | | | | | | | | | | | |
|---|---|---|---|---|---|---|---|---|---|---|---|
| bp: | 500 | 750 | 1,000 | 1,250 | 1,500 | 1,750 | 2,000 | 2,250 | 2,500 | 2,750 | 3,000 |
| $ | 89 | 129 | 149 | 209 | 249 | 289 | 329 | 399 | 449 | 499 | 549 |

An alternate method to the use of gBlock DNA fragments for RNA synthesis includes use of RT-PCR with a forward primer containing a promoter and appropriate reverse primer to amplify a cDNA from a given mRNA present in a biological sample. The cDNA may need to be purified and sequenced to ensure the anticipated product was in hand for its intended use. RNA can be synthesized from the recovered cDNA, 5′ CAP and poly A tail added. Using this RT-PCR based approach also assumes availability of the desired RNA, which may be difficult in certain experimental settings such as study of extinct or rare species, study of cells or organs not readily available, such as human brain tissue or other human vital organs. Other limitations of RT-PCR may include study of hybrid or other proteins forms that do not exist in nature. Of course, there is the added cost of the thermal cycler as well as reagents to perform the RT-PCR reactions. We have performed a version of this method by designing PCR primers to amplify new DNA from the gBlock DNA fragments, using the newly synthesized DNA for in vitro transcription followed by addition of a 5′ CAP and poly (A) tail and found this a satisfactory approach. Another alternate method is chemical synthesis of the desired RNA followed by addition of the 5′ CAP and poly (A) tail to produce the desired synthetic mRNA. However, there are limitations to this method because of costs and lengths of RNA strands that can be synthesized using currently available technology.

In general terms, addition of the 5′ CAP reduces mRNA degradation and aids binding of mRNAs to the ribosome. Similarly, the poly (A) tail reduces mRNA degradation and allows export of mRNAs to the cytoplasm and stimulates protein translation. We find no detectable translation of synthetic mRNAs in the absence of the 5′ CAP and poly (A) tail. In addition, eukaryotic cells express sensors termed pattern recognition receptors or PRRs, that detect pathogen-associated molecular patterns, termed PAMPs. One of these endogenous sensors present in the cytosol is the DExD/H-box helicase, RIG-I, that recognizes the 5′ triphosphate present on nascent cytosolic single- and double-stranded RNAs produced after infection by RNA viruses as part of their normal life cycle and triggers a strong inflammatory response mediated by activation of pro-inflammatory transcription factors, IRFs, and NF-kB. Thus, addition of the 5′ CAP to synthetic mRNAs prior to introduction into the cytosol also prevents activation of these strong inflammatory responses.

We clearly show that the synthetic mRNA system described here has advantages in terms of cost, ease of synthesis, and required time commitment versus standard plasmid-cloning methods for production of mRNAs and functional proteins in human cells. Other advantages can include ability to easily synthesize mRNA sequence variants for structure-function studies or ability to express proteins for study when mRNA is not readily available for plasmid-based cloning or all that is known is the DNA sequence, such as from an extinct species. We did not totally optimize all facets of this synthetic mRNA system to maximize, for example, protein production, so direct comparison between yields of protein using this synthetic mRNA system to more traditional systems is not possible, as this was not the primary goal. Further optimization to maximize mRNA stability and protein production and increase overall systems efficiencies using this system is warranted given the increasing importance of in vitro transcription techniques to lab-based research as well as use of mRNAs as therapeutics or in vaccine development.

### D. REFERENCES

Banerjee A K. 5′-terminal cap structure in eucaryotic messenger ribonucleic acids. Microbiol Rev, 44(2), 175-205 (1980).
Barrett L W, Fletcher S, Wilton S D. Regulation of eukaryotic gene expression by the untranslated gene regions and other non-coding elements. Cell Mol Life Sci, 69(21), 3613-3634 (2012).
Berraondo P, Martini P G V, Avila M A, Fontanellas A. Messenger RNA therapy for rare genetic metabolic diseases. Gut, 68(7), 1323-1330 (2019).
Brown J E, Klement J F, McAllister W T. Sequences of three promoters for the bacteriophage SP6 RNA polymerase. Nucleic Acids Res, 14(8), 3521-3526 (1986).
Chu C, Das K, Tyminski J R et al. Structure of the guanylyltransferase domain of human mRNA capping enzyme. Proc Natl Acad Sci USA, 108(25), 10104-10108 (2011).
Fritsch E F, Sambrook J, Maniatis T. Molecular cloning: A laboratory Manual (Cold Spring Harbor Laboratory Press, 1982).
Gibbons H R, Shaginurova G, Kim L C, Chapman N, Spurlock C F, 3rd, Aune T M. Divergent lncRNA GATA3-AS1 Regulates GATA3 Transcription in T-Helper 2 Cells. Front Immunol, 9, 2512 (2018).
Goubau D, Schlee M, Deddouche S et al. Antiviral immunity via RIG-I-mediated recognition of RNA bearing 5′-diphosphates. Nature, 514(7522), 372-375 (2014).
Guhaniyogi J, Brewer G. Regulation of mRNA stability in mammalian cells. Gene, 265(1-2), 11-23 (2001).
Hadas Y, Sultana N, Youssef E et al. Optimizing Modified mRNA In Vitro Synthesis Protocol for Heart Gene Therapy. Mol Ther-Meth Clin D, 14, 300-305 (2019).
Heinrich M J, Purcell C A, Pruijssers A J et al. Endogenous double-stranded Alu RNA elements stimulate IFN-responses in relapsing remitting multiple sclerosis. J Autoimmun, 100, 40-51 (2019).
Janeway C A, Jr., Medzhitov R. Innate immune recognition. Annu Rev Immunol, 20, 197-216 (2002).
Kadekar S, Nawale G N, Karlsson K, Alander C, Oommen O P, Varghese O P. Synthetic Design of Asymmetric miRNA with an Engineered 3′ Overhang to Improve Strand Selection. Mol Ther-Nucl Acids, 16, 597-604 (2019).
Kozak M. The scanning model for translation: an update. J Cell Biol, 108(2), 229-241 (1989).
Kwon H, Kim M, Seo Y et al. Emergence of synthetic mRNA: In vitro synthesis of mRNA and its applications in regenerative medicine. Biomaterials, 156, 172-193 (2018).
Le T T, Andreadakis Z, Kumar A et al. The COVID-19 vaccine development landscape. Nat Rev Drug Discov, 19(5), 305-306 (2020).
Lodish H, Berk A, A. K C et al. Molecular Cell Biology (Freeman, W.H & Company, 1986).
Mosmann T R, Coffman R L. TH1 and TH2 cells: different patterns of lymphokine secretion lead to different functional properties. Annu Rev Immunol, 7, 145-173 (1989).
O'Shea J J, Paul W E. Mechanisms underlying lineage commitment and plasticity of helper CD4+ T cells. Science, 327(5969), 1098-1102 (2010).
Pardi N, Hogan M J, Porter F W, Weissman D mRNA vaccines—a new era in vaccinology. Nat Rev Drug Discov, 17(4), 261-279 (2018).
Pradere U, Brunschweiger A, Gebert L F R, Lucic M, Roos M, Hall J. Chemical Synthesis of Mono- and Bis-Labeled Pre-MicroRNAs. Angew Chem Int Edit, 52(46), 12028-12032 (2013).
Richter J D. Cytoplasmic polyadenylation in development and beyond. Microbiol Mol Biol Rev, 63(2), 446-456 (1999).
Shatkin A J. Capping of eucaryotic mRNAs. Cell, 9(4 PT 2), 645-653 (1976).
Wen Y, Yue Z, Shatkin A J. Mammalian capping enzyme binds RNA and uses protein tyrosine phosphatase mechanism. Proc Natl Acad Sci USA, 95(21), 12226-12231 (1998).
Wilson J T, Keller S, Manganiello M J et al. pH-Responsive Nanoparticle Vaccines for Dual-Delivery of Antigens and Immunostimulatory Oligonucleotides. Acs Nano, 7(5), 3912-3925 (2013).
Wu X, Brewer G. The regulation of mRNA stability in mammalian cells: 2.0. Gene, 500(1), 10-21 (2012).
Wurdinger T, Badr C, Pike L et al. A secreted luciferase for ex vivo monitoring of in vivo processes. Nat Methods, 5(2), 171-173 (2008).
Zhang C L, Maruggi G, Shan H, Li J W. Advances in mRNA Vaccines for Infectious Diseases. Frontiers in Immunology, 10 (2019).
Zhang G, Gurtu V, Kain S R. An enhanced green fluorescent protein allows sensitive detection of gene transfer in mammalian cells. Biochem Biophys Res Commun, 227(3), 707-711 (1996).
Zhu J, Yamane H, Paul W E. Differentiation of effector CD4 T cell populations (*). Annu Rev Immunol, 28, 445-489 (2010).

### E. SEQUENCES

Nucleic acid sequence for the Secreted Luciferase
SEQ ID NO: 1
ATTTAGGTGACACTATAGAATGACCCTGCTTGCTCAACTCTACGTCTTTGTTTCGTTTTCTGTTCTGCGCCGTT
ACAGATCCAAGCTGTGACCGGCGCCTACCTGAGATCACCGGTCACCATGGAAATCAAGGTGCTGTTTGCCCTCA
TCTGTATTGCTGTTGCTGAGGCAAAACCCACTGAAATCAATGAAGACCTCAATATAGCTGCTGTGGCCTCCAAC
TTTGCCACCACAGATCTTGAGACTGACCTGTTCACCAACTGGGAGACCATGAATGTGATTAGCACTGACACAGA
GCAGGTGAACACAGATGCTGACAGGGGCAAGCTGCCTGGCAAAAAACTCCCCCCAGATGTCCTGAGGGAGCTGG
AGGCCAATGCCAGAAGGGCTGGTTGCACAAGAGGCTGCCTCATTTGCCTCTCCCACATTAAGTGCACCCCTAAG
ATGAAGAAATTTATCCCTGGCAGGTGCCACACTTATGAAGGTGAAAAGGAGTCTGCTCAGGGAGGGATTGGAGA
GGCAATTGTTGATATCCCAGAGATTCCTGGCTTCAAGGATAAGGAGCCACTGGACCAGTTTATTGCTCAAGTGG
ACCTCTGTGCTGATTGCACCACTGGCTGTCTGAAGGGCCTTGCCAATGTCCAGTGCTCTGACCTCCTGAAGAAG
TGGCTTCCCCAGAGGTGTACCACTTTTGCCAGCAAGATTCAGGGTAGGGTGGACAAAATCAAGGGTCTGGCTGG
GGACAGATGATTAGCTAGCTGGCCAGACATGATAAGATACATTGATGAGTTTGGACAAACCACAACTAGAATGC
AGTGAAAAAAATGCTTTATTTGTGAAATTTGTGATGCTATTGCTTTATTTGTAACCATTATAAGCTGCAATAAA
CAAGTTAACAACAACAATTGCATTCATTTTATGTTTCAGGTTCAGGGGGAGGTGTGGGAGGTTTTTTAAAGCAA
GTAAAACCTCTACAAATGTGGTATGGAAAAAAAAAA
Nucleic acid sequence for eGFP
SEQ ID NO: 2
ATTTAGGTGACACTATAGAATGACCCTGCTTGCTCAACTCTACGTCTTTGTTTCGTTTTCTGTTCTGCGCCGTT
ACAGATCCAAGCTGTGACCGGCGCCTACCTGAGATCACCGGTCACCATGGTGAGCAAGGGCGAGGAGCTGTTCA
CCGGGGTGGTGCCCATCCTGGTCGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGGCAAGCTGCCCGTGCCCTGGCC
CACCCTCGTGACCACCCTGACCTACGGCGTGCAGGCTTCAGCCGCTACCCCGACCACATGAAGCAGCACGACTT
CTTCAAGTCCGCCATGCCCGAAGGCTACGTCCAGGAGCGCACCATCTTCTTCAAGGACGACGGCAACTACAAGA
CCCGCGCCGAGGTGAAGTTCGAGGGCGACACCCTGGTGAACCGCATCGAGCTGAAGGGCATCGACTTCAAGGAG
GACGGCAACATCCTGGGGCACAAGCTGGAGTACAACTACAACAGCCACAACGTCTATATCATGGCCGACAAGCA
GAAGAACGGCATCAAGGTGAACTTCAAGATCCGCCACAACATCGAGGACGGCAGCGTGCAGCTCGCCGACCACT
ACCAGCAGAACACCCCCATCGGCGACGGCCCCGTGCTGCTGCCCGACAACCACTACCTGAGCACCCAGTCCGCC
CTGAGCAAAGACCCCAACGAGAAGCGCGATCACATGGTCCTGCTGGAGTTCGTGACCGCCGCCGGGATCACTCT
CGGCATGGACGAGCTGTACAAGTAATTAGCTAGCTGGCCAGACATGATAAGATACATTGATGAGTTTGGACAAA
CCACAACTAGAATGCAGTGAAAAAAATGCTTTATTTGTGAAATTTGTGATGCTATTGCTTTATTTGTAACCATT
ATAAGCTGCAATAAACAAGTTAACAACAACAATTGCATTCATTTTATGTTTCAGGTTCAGGGGGAGGTGTGGGA
GGTTTTTTAAAGCAAGTAAAACCTCTACAAATGTGGTATGGAAAAAAAAAA
Nucleic acid sequence for IL-4
SEQ ID NO: 3
ATTTAGGTGACACTATAGAATGACCCTGCTTGCTCAACTCTACGTCTTTGTTTCGTTTTCTGTTCTGCGCCGTT
ACAGATCCAAGCTGTGACCGGCGCCTACCTGAGATCACCGGTCACCATGGGTCTCACCTCCCAACTGCTTCCCC
CTCTGTTCTTCCTGCTAGCATGTGCCGGCAACTTTGTCCACGGACACAAGTGCGATATCACCTTACAGGAGATC
ATCAAAACTTTGAACAGCCTCACAGAGCAGAAGACTCTGTGCACCGAGTTGACCGTAACAGACATCTTTGCTGC
CTCCAAGAACACAACTGAGAAGGAAACCTTCTGCAGGGCTGCGACTGTGCTCCGGCAGTTCTACAGCCACCATG
AGAAGGACACTCGCTGCCTGGGTGCGACTGCACAGCAGTTCCACAGGCACAAGCAGCTGATCCGATTCCTGAAA
CGGCTCGACAGGAACCTCTGGGGCCTGGCGGGCTTGAATTCCTGTCCTGTGAAGGAAGCCAACCAGAGTACGTT
GGAAAACTTCTTGGAAAGGCTAAAGACGATCATGAGAGAGAAATATTCAAAGTGTTCGAGCTGATAGCTAGCTG
GCCAGACATGATAAGATACATTGATGAGTTTGGACAAACCACAACTAGAATGCAGTGAAAAAAATGCTTTATTT
GTGAAATTTGTGATGCTATTGCTTTATTTGTAACCATTATAAGCTGCAATAAACAAGTTAACAACAACAATTGC
ATTCATTTTATGTTTCAGGTTCAGGGGGAGGTGTGGGAGGTTTTTTAAAGCAAGTAAAACCTCTACAAATGTGG
TATGGAAAAAAAAAA
Nucleic acid sequence for IL-12A
SEQ ID NO: 4
ATTTAGGTGACACTATAGAATGACCCTGCTTGCTCAACTCTACGTCTTTGTTTCGTTTTCTGTTCTGCGCCGTT
ACAGATCCAAGCTGTGACCGGCGCCTACCTGAGATCACCGGTCACCATGTGGCCCCCTGGGTCAGCCTCCCAGC
CACCGCCCTCACCTGCCGCGGCCACAGGTCTGCATCCAGCGGCTCGCCCTGTGTCCCTGCAGTGCCGGCTCAGC
ATGTGTCCAGCGCGCAGCCTCCTCCTTGTGGCTACCCTGGTCCTCCTGGACCACCTCAGTTTGGCCAGAAACCT
CCCCGTGGCCACTCCAGACCCAGGAATGTTCCCATGCCTTCACCACTCCCAAAACCTGCTGAGGGCCGTCAGCA
ACATGCTCCAGAAGGCCAGACAAACTCTAGAATTTTACCCTTGCACTTCTGAAGAGATTGATCATGAAGATATC
ACAAAAGATAAAACCAGCACAGTGGAGGCCTGTTTACCATTGGAATTAACCAAGAATGAGAGTTGCCTAAATTC
CAGAGAGACCTCTTTCATAACTAATGGGAGTTGCCTGGCCTCCAGAAAGACCTCTTTTATGATGGCCCTGTGCC
TTAGTAGTATTTATGAAGACTTGAAGATGTACCAGGTGGAGTTCAAGACCATGAATGCAAAGCTTCTGATGGAT
CCTAAGAGGCAGATCTTTCTAGATCAAAACATGCTGGCAGTTATTGATGAGCTGATGCAGGCCCTGAATTTCAA
CAGTGAGACTGTGCCACAAAAATCCTCCCTTGAAGAACCGGATTTTTATAAAACTAAAATCAAGCTCTGCATAC
TTCTTCATGCTTTCAGAATTCGGGCAGTGACTATTGATAGAGTGATGAGCTATCTGAATGCTTCCTAATAGCTA
GCTGGCCAGACATGATAAGATACATTGATGAGTTTGGACAAACCACAACTAGAATGCAGTGAAAAAAATGCTTT
ATTTGTGAAATTTGTGATGCTATTGCTTTATTTGTAACCATTATAAGCTGCAATAAACAAGTTAACAACAACAA
TTGCATTCATTTTATGTTTCAGGTTCAGGGGGAGGTGTGGGAGGTTTTTTAAAGCAAGTAAAACCTCTACAAAT
GTGGTATGGAAAAAAAAAA
Nucleic acid sequence for IL-12B
SEQ ID NO: 5
ATTTAGGTGACACTATAGAATGACCCTGCTTGCTCAACTCTACGTCTTTGTTTCGTTTTCTGTTCTGCGCCGTT
ACAGATCCAAGCTGTGACCGGCGCCTACCTGAGATCACCGGTCACCATGTGTCACCAGCAGTTGGTCATCTCTT
GGTTTTCCCTGGTTTTTCTGGCATCTCCCCTCGTGGCCATATGGGAACTGAAGAAAGATGTTTATGTCGTAGAA
TTGGATTGGTATCCGGATGCCCCTGGAGAAATGGTGGTCCTCACCTGTGACACCCCTGAAGAAGATGGTATCAC
CTGGACCTTGGACCAGAGCAGTGAGGTCTTAGGCTCTGGCAAAACCCTGACCATCCAAGTCAAAGAGTTTGGAG
ATGCTGGCCAGTACACCTGTCACAAAGGAGGCGAGGTTCTAAGCCATTCGCTCCTGCTGCTTCACAAAAAGGAA
GATGGAATTTGGTCCACTGATATTTTAAAGGACCAGAAAGAACCCAAAAATAAGACCTTTCTAAGATGCGAGGC
CAAGAATTATTCTGGACGTTTCACCTGCTGGTGGCTGACGACAATCAGTACTGATTTGACATTCAGTGTCAAAA
GCAGCAGAGGCTCTTCTGACCCCCAAGGGGTGACGTGCGGAGCTGCTACACTCTCTGCAGAGAGAGTCAGAGGG
GACAACAAGGAGTATGAGTACTCAGTGGAGTGCCAGGAGGACAGTGCCTGCCCAGCTGCTGAGGAGAGTCTGCC
CATTGAGGTCATGGTGGATGCCGTTCACAAGCTCAAGTATGAAAACTACACCAGCAGCTTCTTCATCAGGGACA
TCATCAAACCTGACCCACCCAAGAACTTGCAGCTGAAGCCATTAAAGAATTCTCGGCAGGTGGAGGTCAGCTGG
GAGTACCCTGACACCTGGAGTACTCCACATTCCTACTTCTCCCTGACATTCTGCGTTCAGGTCCAGGGCAAGAG
CAAGAGAGAAAAGAAAGATAGAGTCTTCACGGACAAGACCTCAGCCACGGTCATGCAAGAGAGAAAAGAAAGAT
AGAGTCTTCACGGACAAGACCTCAGCCACGGTCATCTGCCGCAAAAATGCCAGCATTAGCGTGCGGGCCCAGGA
CCGCTACTATAGCTCATCTTGGAGCGAATGGGCATCTGTGCCCTGCAGTTAGTAGCTAGCTGGCCAGACATGAT
AAGATACATTGATGAGTTTGGACAAACCACAACTAGAATGCAGTGAAAAAAATGCTTTATTTGTGAAATTTGTG
ATGCTATTGCTTTATTTGTAACCATTATAAGCTGCAATAAACAAGTTAACAACAACAATTGCATTCATTTTATG
TTTCAGGTTCAGGGGGAGGTGTGGGAGGTTTTTTAAAGCAAGTAAAACCTCTACAAATGTGGTATGGAAAAAAA
AAA
RED: SP6 PROMOTER
Green 5′UTR
TEAL: KOZACK SEQ
BLUE: START/STOP
BLACK: LUCIA GENE
SV40 pAn (3′UTR)
ORANGE: POLY A TAIL
Consensus Kozak nucleic acid sequence
SEQ ID NO: 6
gccRccATGG
where R can be A or G
Kozak nucleic acid sequence
SEQ ID NO: 7
CCGGTCACCATG
T7 promoter
SEQ I NO: 8
TAATACGACTCACTATAGGGAGA
T3 promoter
SEQ ID NO: 9
AATTAACCCTCACTAAAGGGAGA
K11 promoter
SEQ ID NO: 10
AATTAGGGCACACTATAGGGAGA
SP6
SEQ ID NO: 11
ATTTACGACACACTATAGAAGAA

1. A method of making a synthetic ribonucleic acid (RNA) strand, the method comprising obtaining a double stranded (ds) deoxyribonucleic acid (DNA) comprising a nucleic acid of interest and transcribing RNA from the dsDNA in vitro.

2. The method of making a synthetic RNA strand of claim 1, further comprising adding a 5′ CAP to the transcribed RNA.

3. The method of making a synthetic RNA strand of claim 1, further comprising adding a poly Adenosine (polyA) tail to the 3′ end of the transcribed RNA.

4. The method of making a synthetic RNA strand of claim 1, wherein the dsDNA comprises in order from 5′ to 3′ an RNA promoter sequence, a 5′ untranslated region (UTR), a Kozack sequence, the nucleic acid of interest, and a 3′ UTR.

5. The method of making a synthetic RNA strand of claim 4, wherein the Kozack sequence comprises the sequence CCGGTCACCATG or GCCRCCATGG.

6. The method of making a synthetic RNA strand of claim 4, wherein the RNA promoter sequence is a DNA dependent RNA polymerase promoter from a bacteriophage.

7. The method of making a synthetic RNA strand of claim 6, wherein the DNA dependent RNA promoter comprises a T7 promoter, T3 promoter, SP6 promoter, or KII promoter.

8. The method of making a synthetic RNA strand of claim 1, wherein the nucleic acid of interest is between 100 and 10,000 base pairs in length.

9. A method of making an exogenous protein from a synthetic deoxyribonucleic acid (DNA) comprising obtaining a double stranded (ds) deoxyribonucleic acid (DNA) comprising a nucleic acid of interest, transcribing ribonucleic acid (RNA) from the dsDNA in vitro, and transfecting a cell with the transcribed RNA; wherein the transfected RNA is expressed by the cell.

10. The method of making an exogenous protein of claim 9, further comprising adding a 5′ CAP to the transcribed RNA prior to transfection.

11. The method of making an exogenous protein of claim 9, further comprising adding a poly Adenosine (polyA) tail to the 3′ end of the transcribed RNA prior to transfection

12. The method of making an exogenous protein of claim 9, wherein the dsDNA comprises in order from 5′ to 3′ an RNA promoter sequence, a 5′ untranslated region (UTR), a Kozack sequence, the nucleic acid of interest, and a 3′ UTR.

13. The method of making an exogenous protein of claim 12, wherein the Kozack sequence comprises the sequence CCGGTCACCATG or GCCRCCATGG.

14. The method of making an exogenous protein of claim 12, wherein the RNA promoter sequence is a DNA dependent RNA polymerase promoter from a bacteriophage.

15. The method of making an exogenous protein of claim 14, wherein the DNA dependent RNA promoter comprises a T7 promoter, T3 promoter, SP6 promoter, or KII promoter.

16. The method of making an exogenous protein of claim 9, wherein the nucleic acid of interest is between 100 and 10,000 base pairs in length.
